(19) United States
(12) Patent Application Publication
(10) Pub. No.: US 2019/0110472 A1
(43) Pub. Date: Apr. 18, 2019

(54) ACTIVE INGREDIENT COMPOSITIONS COMPRISING N-ALKENOYL-N-ALKYLGLUCAMIDES AND THE USE THEREOF

(71) Applicant: Clariant International, Ltd., Muttenz 1 (CH)

(72) Inventors: Martin BAUER, Frankfurt am Main (DE); Peter BAUR, Schondorf (DE)

(73) Assignee: Clariant International, Ltd., Muttenz 1 (CH)

(21) Appl. No.: 15/782,560

(22) Filed: Oct. 12, 2017

(51) Int. Cl. A01N 37/18; C07C 233/60

(52) U.S. Cl. CPC A01N 37/18; A01N 25/06; C07C 233/60

(57) ABSTRACT

The invention relates to active ingredient compositions comprising
a) one or more active substances, and
b) one or more N-alkenoyl-N-alkylglucamides of the formula I

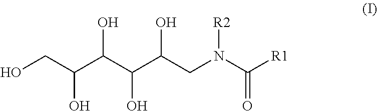

in which
R1 is a linear or branched alkenyl group which has 7 to 16 carbon atoms and has a non-terminal double bond or a plurality of conjugated or non-conjugated double bonds, and
R2 is an alkyl group having 1 to 4 carbon atoms.

The compounds of formula I can be used for reducing drift and for improving wettability when appyling active ingredient compositions.

Active ingredient compositions comprising N-alkenoyl-N-alkylglucamides and the use thereof.

The invention relates to novel active ingredient compositions comprising unsaturated N-alkenoyl-N-alkylglucamides, such as crop treatment compositions. The novel active ingredient compositions can be used for production of stable spray liquid liquids and the unsaturated N-alkenoyl-N-alkylglucamides are self-emulsifying therein. They have a marked tendency to reduce the dynamic surface tension and hence excellent wetting characteristics, and bring about a reduction in the fine droplet content in the spraying of the spray liquid. Moreover, they do not lead to enhanced uptake of the active ingredients and are therefore especially suitable for active ingredient formulations comprising contact pesticides or soil-active pesticides.

Crop protection compositions are applied to agricultural production fields in a very efficient manner using spray tanks in aircraft, tractors or other equipment. In order to achieve very exact positioning of the active substances, it is necessary to obtain a very narrow spray cone and to prevent drift of the spray away from the target site.

The drift of the spray is determined essentially by the droplet size distribution. The smaller the droplets, the longer the residence time in the air and the greater their tendency to drift horizontally and to evaporate and/or to miss the target site. The literature discloses that the fine droplet fraction of <150 μm (Teske et al., 2004, The Role of Small Droplets in Classifying Drop Size Distributions, IASS Americas 17th Annual Conference, Arlington Va.), especially <100 μm (Vermeer et al., Proc. ISAA 2013, The use of adjuvanted formulations for drift control) determines the proportion of droplets in the spray that contributes to the drift effect. The reduction of the fine droplet content in the spray is therefore crucial to reduction of drift and is therefore utilized for determination of the drift properties of a composition.

A marked minimization of the drift effect can be achieved by addition of suitable “drift control agents” to crop protection formulations, and these lead to a decrease in the fine droplet content and hence an increase in droplet size in the spray. The formulations modified with “drift control agents” additionally have to be insensitive to the shear forces to which they are exposed in the spray pumps and nozzles. Good biodegradability, compatibility with other constituents of the crop protection compositions and high storage stability and thermal stability are further requirements for “drift control agents”. It is known that the rheology of aqueous compositions can be modified by addition of water-soluble polymers, for example polyacrylamides, acrylamide/acrylic acid polymers, sodium polyacrylate, carboxymethyl cellulose, hydroxyethyl cellulose, methyl cellulose, polysaccharides, natural and synthetic guar gum (U.S. Pat. No. 4,413,087, U.S. Pat. No. 4,505,827, U.S. Pat. No. 5,874,096), leading to a shift in the droplet size spectrum toward larger droplets.

Molasses and organic thickeners have also been described as effective drift reduction agents (Pesticide Drift III; Drift Reduction with Spray Thickeners; Ware, G. W. et al.; J. of Economic Entomology 63; 1314-1316; 1970). It is additionally known that particular emulsions—via a mechanism which is not fully understood—lead to a reduced fine droplet content (Vermeer et al.; Crop Protection 44; 2013; Spray drift review: The extent to which a formulation can contribute to spray drift reduction).

The use of sugar-based surfactants such as alkyl-N-methylglucosamides, for example in cleaning compositions and cosmetic products, is described in the literature (F. W. Lichtenthaler, “Carbohydrates as Organic Raw Materials” in Ullmann's Encyclopedia of Industrial Chery, Wiley-VCH Verlag, 2010).

WO 96/16540 describes pesticide compositions which comprise long-chain alkylamides which bear a polyhydroxycarbonyl substituent having at least three hydroxyl groups on the amide nitrogen.

WO 2014/067663 describes aqueous adjuvant compositions which comprise one or more alkylglucamides having a linear or branched C5-C9-alkyl group and are suitable for enhancing the action of pesticides and for production of aqueous pesticide compositions.

US 2017/0218293 A1, DE 10 2014 003 367 Al and DE 10 2014 003 215 Al disclose N-methyl-N-acylglucamides as corrosion inhibitors. These compounds possess an acyl group having 7 to 21 carbon atoms which can be saturated or unsaturated.

US 2015/0126616 A1 discloses N-methyl-N-acylglucamides as thickening agents in surfactant solutions. The N-methyl-N-acylglucamides used are a mixture of different compounds at least 60% thereof having unsaturated acyl groups of 12, 14 or 18 carbon atoms and less than 5% thereof having acyl groups shorter than 12 carbon atoms.

WO 2016/092030 discloses sugar surfactants containing a dec-9-enoyl group. These sugar surfactants thus have an ethylenically unsaturated radical having a terminal double bond. The alkylglucamides described in this document are suitable for enhancing the action of pesticides and for producing aqueous pesticide compositions.

Even though good results are already achieved with the known systems, there is still a need, for technical, economic and ecological reasons, to find suitable “drift control agents” which, even under practical conditions, effectively increase the droplet volumes of the aqueous compositions and reduce drift of the spray . Especially the antagonism with crop treatment composition formulations, fertilizers and interface-active tankmix additives limits the effect of the “drift control agents”, and more robust products are needed.

It has now been found that, surprisingly, selected unsaturated alkylglucamides are suitable as drift-reducing adjuvants for active ingredient formulations and when sprayed, leading to an effective increase in droplet size through a reduction in the fine droplet content in the spray. Furthermore, these adjuvants show a marked improvement in the wetting characteristics of the active ingredient formulations. For wetting agents it is highly unsual that they also exhibit drift reducting properties. Most of the times wetting agents with strong surface tension reduction have a negative effect on the drift reducing characteristics of a formulation. Furthermore, these adjuvants are readily incorporable into the active ingredient formulations and spray liquids, since they are self-emulsifying. Moreover, they do not lead to enhanced uptake of the active ingredients and are therefore especially suitable for active ingredient formulations comprising contact pesticides or soil-active pesticides.

The invention provides active ingredient compositions comprising
a) one or more active substances, and
b) one or more N-alkenoyl-N-alkylglucamides of the formula I

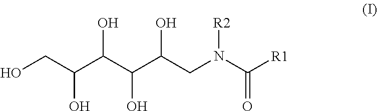

in which
R1 is a linear or branched alkenyl group which has 7 to 16 carbon atoms and has a non-terminal double bond or a plurality of conjugated or non-conjugated double bonds, and
R2 is an alkyl group having 1 to 4 carbon atoms.

The N-alkenoyl-N-alkylglucamides of the formula I are prepared, for example, by reacting the corresponding sugar amines obtainable from reductive sugars, such as N-methylglucamine, with commercially available unsaturated N-alkenyl acid methyl esters. A corresponding preparation method is described, for example, in EP-A 0 550 637.

In the N-alkenoyl-N-alkylglucamides of the formula I, the R1 radical is a linear or branched alkenyl group which has 7 to 16 carbon atoms and a non-terminal double bond or two or more conjugated or non-conjugated double bonds. The R1 radicals derive, for example, from alkenyl acid methyl esters.

The double bonds of the R1 radicals may be present in cis and/or trans configuration.

If two or more double bonds are present in an R1 radical, these are generally separated by an alkylene group, especially by a methylene group. However, R1 radicals derived from conjugated fatty acids where the double bonds are closer to one another, namely in conjugated form, are also possible.

Preferably used are N-alkenoyl-N-alkylglucamides of the formula I in which the R1 radical is a linear or branched alkenyl group which has 11 to 15 carbon atoms and a non-terminal double bond or two or more conjugated or non-conjugated double bonds.

Very particularly used are N-alkenoyl-N-alkylglucamides of the formula I in which the R1 radical is a linear or branched alkenyl group which has 12 to 14 carbon atoms and a non-terminal double bond or two or more conjugated or non-conjugated double bonds.

Preferably used are N-alkenoyl-N-alkylglucamides of the formula I in which R1 is a dodecadienyl radical or a tetradecadienyl radical, and especially a 8,11-dodecadienyl radical or a 8,11 tetradecadienyl radical. These preferred radicals derive from 9,12 tridecadienoic acids or from 9,12 pentadecadienoic acids or esters thereof, such as the methyl esters.

Most preferably used are N-alkenoyl-N-alkylglucamides of the formula I in which R1 is an undecenyl radical having a non-terminal double bond, and especially an undec-8-enyl radical. These preferred radicals derive from dodecenoic acids having a non-terminal double bond or esters thereof, such as the methyl esters, especially from dodec-9-enyl acid methyl ester.

Further examples of most preferably used N-alkenoyl-N-alkylglucamides of the formula I are those in which R1 is polyethylenically unsaturated alkenyl groups having 11 to 15 carbon atoms, preferably having 12 to 14 carbon atoms, the double bonds of which are non-conjugated or conjugated. Preferably, polyunsaturated alkenyl groups R1 do not have a double bond on the terminal carbon atom. Preferably, polyunsaturated alkenyl groups have two or three double bonds.

N-Alkenoyl-N-alkylglucamides of the formula (I) may also take the form of mixtures having different R1 and/or R2 radicals, or these mixtures may include some alkylglucamides in which some of the R1 radicals have been replaced by a linear or branched alkyl group, for example alkyl groups having 7 to 19 carbon atoms.

Likewise particularly preferably used are N-alkenoyl-N-alkylglucamides of the formula I that have been prepared from a mixture of N-alkenyl acid methyl esters that comprises diunsaturated C13-alkenyl acid methyl ester, monounsaturated C14-alkenyl acid methyl ester, monounsaturated C15-alkenyl acid methyl ester and diunsaturated C15-alkenyl acid methyl ester.

The particularly preferably used N-alkenoyl-N-alkylglucamides of the formula I of this type especially include those in which R1 is a mixture of several monounsaturated C₁₂-C₁₄-alkenyl groups having a non-terminal double bond which may optionally also contain one or more diunsaturated C₁₂-C₁₄-alkenyl groups.

The proportion of N-alkenoyl-N-alkylglucamides of the formula I with C12-alkenyl radicals R1 containing two double bonds in this mixture is preferably 1% to 50% by weight, more preferably 10% to 40% by weight and most preferably 15% to 30% by weight. The proportion of N-alkenoyl-N-alkylglucamides of the formula I with C13-alkenyl radicals R1 containing one non-terminal double bond in this mixture is preferably 1% to 30% by weight, more preferably 2% to 20% by weight and most preferably 5% to 15% by weight. The proportion of N-alkenoyl-N-alkylglucamides of the formula I with C14-alkenyl radicals R1 containing one non-terminal double bond in this mixture is preferably 1% to 50% by weight, more preferably 10% to 40% by weight and most preferably 20% to 30% by weight. And the proportion of N-alkenoyl-N-alkylglucamides of the formula I with C14-alkenyl radicals containing two double bonds in this mixture is preferably 1% to 50% by weight, more preferably 10% to 40% by weight and most preferably 15% to 30% by weight.

The R2 radical in formula I is a C₁-C₄-alkyl group, for example methyl, ethyl, n-propyl, isopropyl, n-butyl or isobutyl. Preferably, R2 is methyl.

The pentahydroxyhexyl radical in the N-alkenoyl-N-alkylglucamides of the formula I possesses various chiral centers, and so a plurality of stereoisomers may exist in each case. Typically, the N-alkenoyl-N-alkylglucamides of the formula I are prepared from naturally occurring sugars, such as D-glucose, but the use of other natural or synthetic hexoses or other C₆ units is also possible in principle, such that different stereoisomers of the formula I can result.

The N-alkenoyl-N-alkylglucamides of the formula I feature an advantageous toxicological and ecological profile. They have low solubility in water, but are self-emulsifying in water and form stable homogeneous emulsions.

The invention also relates to N-alkenoyl-N-alkylglucamide of the formula I

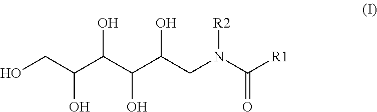

in which
R1 is an undec-8-enyl radical or wherein R1 derives from a mixture of N-alkenyl acid methyl esters that comprises diunsaturated C13-alkenyl acid methyl esters, monounsaturated C14-alkenyl acid methyl esters, monounsaturated C15-alkenyl acid methyl esters and diunsaturated C15-alkenyl acid methyl esters, and
R2 is an alkyl group having 1 to 4 carbon atoms.

Furthermore, the invention provides adjuvant compositions comprising
(A1) an N-alkenoyl-N-alkylglucamide of the formula I, in which R1 is an undec-8-enyl radical or wherein R1 derives from a mixture of N-alkenyl acid methyl esters that comprises diunsaturated C13-alkenyl acid methyl esters, monounsaturated C14-alkenyl acid methyl esters, monounsaturated C15-alkenyl acid methyl esters and diunsaturated C15-alkenyl acid methyl esters, and
R2 is an alkyl group having 1 to 4 carbon atoms, and
(A2) a cosolvent.

The proportion of the N-alkenoyl-N-alkylglucamides of the formula I (A1) in the adjuvant composition is typically 5% to 99.9% by weight, preferably 50% to 99% by weight and more preferably 85% to 95% by weight, based on the total amount of the adjuvant composition.

The cosolvent (A2) may either be present as a secondary component from the preparation process for the N-alkenoyl-N-alkylglucamide and/or have been added subsequently to the adjuvant composition. The cosolvent may be a single solvent or a mixture of two or more solvents. Suitable solvents for this purpose are all polar solvents that are compatible with the aqueous composition and form a homogeneous phase. Suitable cosolvents are, for example, monohydric alcohols such as methanol, ethanol, propanols, butanols, benzyl alcohol or polyhydric alcohols such as ethylene glycol, diethylene glycol, propylene glycol or glycerol, or polyglycols such as polyethylene glycols, polypropylene glycols or mixed polyalkylene glycols (PAGs). Further suitable solvents are ethers, for example propylene glycol mono- or dimethyl ether, dipropylene glycol mono- or dimethyl ether, amides, for example N-methyl- or N-ethylpyrrolidone, N,N-dimethyllactamide, -caprylamide or -decanamide.

Particularly suitable cosolvents are mono- or polyhydric alcohols, and especially suitable cosolvents are di- or trihydric alcohols such as propylene glycol, glycerol or polyethylene glycols, polypropylene glycols or mixed polyalkylene glycols (PAGs).

Most preferably, the adjuvant compositions comprise, as component (A2), propylene glycol or dipropylene glycol, and combinations of the two with one another or with polypropylene glycol or polyethylene glycol having up to 10 repeat units. Propylene glycol is especially preferred.

The proportion of cosolvent (A2) in the adjuvant composition is typically 0.1% to 95% by weight, preferably 1% to 50% by weight and more preferably 5% to 15% by weight, based on the total amount of the adjuvant composition.

“Active ingredient compositions” in the context of the invention are understood to mean compositions comprising one or more active substances and one or more compounds of the formula I. The active substances especially include pesticides, phytohormones, preferably growth regulators, biological pesticides, salts deployable in water, preferably fertilizers or plant nutrients or fungicidal copper compounds, and repellents. The aforementioned active substances are referred to in the context of the present invention as “agrochemical substances”.

With the above-described N-alkenoyl-N-alkylglucamides of the formula I, it is possible to produce active ingredient compositions of the invention having excellent performance properties.

The invention also relates to active ingredient compositions comprising one or more active substances and one or more N-alkenoyl-N-alkylglucamides of the formula I, where the one or more active substances are preferably pesticides, and at least one of the one or more pesticides is more preferably a contact pesticide and/or a soil-active herbicide.

In a preferred embodiment of the invention, the one or more N-alkenoyl-N-alkylglucamides of the formula I are in the form of a tankmix additive, meaning that the one or more N-alkenoyl-N-alkylglucamides of the formula I are only added to a spray liquid produced from a concentrated composition directly prior to deployment.

In another preferred embodiment of the invention, the one or more N-alkenoyl-N-alkylglucamides of the formula I are in the form of an in-can variant, meaning that the one or more N-alkenoyl-N-alkylglucamides of the formula I have already been incorporated into a concentrated composition together with the ingredients of the active ingredient composition and deployed as a spray liquid diluted with water.

The one or more N-alkenoyl-N-alkylglucamides of the formula I are preferably present in ready-to-use active ingredient compositions in the form of spray liquids, in which case the amount of the one or more N-alkenoyl-N-alkylglucamides of the formula I in the spray liquid is preferably from 0.001% to 5% by weight, more preferably from 0.005% to 3% by weight, especially preferably from 0.01% to 1% by weight and exceptionally preferably from 0.05% to 0.2% by weight, based in each case on the total weight of the spray liquid. These spray liquids used in the inventive use preferably contain one or more agrochemical substances.

If an active ingredient composition comprises two or more N-alkenoyl-N-alkylglucamides of the formula I, the stated amount is understood to mean the total content of all N-alkenoyl-N-alkylglucamides of the formula I.

The radical definitions, value ranges and elucidations given above, in general terms or in ranges of preference, can be combined with one another as desired, i.e. including combinations between the particular ranges and ranges of preference.

The one or more N-alkenoyl-N-alkylglucamides of the formula I can be used in the production of active ingredient compositions. The result here is compositions used in accordance with the invention that comprise one or more N-alkenoyl-N-alkylglucamides of the formula I and one or more agrochemical substances.

The active substances present in the active ingredient compositions of the invention may be a single active substance or a mixture of two or more active substances. The active substances may generally be any active ingredients used in active ingredient compositions, for example in crop treatment compositions, with which a desired effect can be achieved in the species treated, for example plants.

Preferably, the active substances are one or more pesticides.

“Pesticides” are understood in the context of the present invention to mean herbicides, fungicides, insecticides, acaricides, bactericides, molluscicides, nematicides, plant growth regulators and rodenticides. An overview of the most relevant pesticides can be found, for example, in “The Pesticide Manual” from the British Crop Protection Council, 16^{th} Edition 2012, editor: C. MacBean. Explicit reference is hereby made to the active ingredients listed therein. They are incorporated into this description by citation.

Examples of fungicides include:

(1) Ergosterol biosynthesis inhibitors, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifin, nuarimol, oxpoconazole, paclobutrazole, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, uniconazole-p, viniconazole, voriconazole, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-[1-(4-methoxypjenoxy)-3,3-dimethylbutan-2-yl] 1H-imidazole-1-carbothioate.

(2) Respiration inhibitors (respiratory chain inhibitors), for example bixafen, boscalid, carboxin, diflumetorim, fenfuram, fluopyram, flutolanil, fluxapyroxad, furametpyr, furmecyclox, isopyrazam (mixture of the syn-epimeric racemate 1 RS,4SR,9RS and of the anti-epimeric racemate 1 RS,4SR,9SR), isopyrazam (anti-epimeric racemate), isopyrazam (anti-epimeric enantiomer 1 R,4S,9S), isopyrazam (anti-epimeric enantiomer 1S,4R,9R), isopyrazam (syn-epimeric racemate 1 RS,4SR,9RS), isopyrazam (syn-epimeric enantiomer 1 R,4S,9R), isopyrazam (syn-epimeric enantiomer 1S,4R,9S), mepronil, oxycarboxin, penflufen, penthiopyrad, sedaxane, thifluzamid, 1-methyl-N-[2-(1,1,2,2-tetrafluoroethoxy)phenyl]-3-(trifluoromethyl)-1H-pyrazole-4-carboxamide, 3-(difluoromethyl)-1-methyl-N-[2-(1,1,2,2-tetrafluoroethoxy)phenyl]-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide, 5,8-difluoro-N-[2-(2-fluoro-4-{[4-(trifluoromethyl)pyridin-2-yl]oxy}phenyl)ethyl]quinazoline-4-amine, N-[9-(dichloromethylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide.

(3) Respiration inhibitors (respiratory chain inhibitors) acting on complex III of the respiratory chain, for example ametoctradin, amisulbrom, azoxystrobin, cyazofamid, coumethoxystrobin, coumoxystrobin, dimoxystrobin, enestroburin, famoxadone, fenamidone, fenoxystrobin, fluoxastrobin, kresoxim-methyl, metominostrobin, orysastrobin, picoxystrobin, pyraclostrobin, pyrametostrobin, pyraoxystrobin, pyribencarb, triclopyricarb, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(methoxyimino)-N-methyl-2-(2-{[({(1E)-1-[3-(trifluoromethyl)phenyl]ethylidene}amino)oxy]methyl}phenyl)ethanamide, (2E)-2-(methoxyimino)-N-methyl-2-{2-[(E)-({1-[3-(trifluoromethyl)phenyl[ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino{oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1 E)-1-[3-(trifluoromethyl)phenyl]ethylidene}amino)oxy]methyl}phenyl)-2,4-dihydro-3H-1,2,4-triazol-3-one, methyl (2E)-2-{2-[({cyclopropyl[(4-methoxyphenyl)imino]methyl}sulfanyl)methyl]phenyl}-3-methoxyprop-2-enoate, N-(3-ethyl-3,5,5-trimethylcyclohexyl)-3-(formylamino)-2-hydroxybenzamide, 2-{2-[(2,5-dimethylphenoxy)methyl]phenyl}-2-methoxy-N-methylacetamide and (2R)-2-{2-[(2,5-dimethylphenoxy)methyl]phenyl}-2-methoxy-N-methylacetamide.

(4) Mitosis and cell division inhibitors, for example benomyl, carbendazim, chlorfenazole, diethofencarb, ethaboxam, fluopicolide, fuberidazole, pencycuron, thiabendazole, thiophanate-methyl, thiophanate, zoxamide, 5-chloro-7-(4-methylpiperidin-1-yl)-6-(2,4,6-trifluorophenyl)[1,2,4]triazolo[1,5-a]pyrimidine and 3-chloro-5-(6-chloropyridin-3-yl)-6-methyl-4-(2,4,6-trifluorophenyl)pyridazine.

(5) Compounds with multisite activity, for example Bordeaux mixture, captafol, captan, chlorothalonil, copper preparations such as copper hydroxide, copper naphthenate, copper oxide, copper oxychloride, copper sulfate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulfur and sulfur preparations, for example calcium polysulfide, thiram, tolylfluanid, zineb and ziram.

(6) Resistance inductors, for example acibenzolar-S-methyl, isotianil, probenazole and tiadinil.

(7) Amino acid and protein biosynthesis inhibitors, for example andoprim, blasticidin-S, cyprodinil, kasugamycin, kasugamycin hydrochloride hydrate, mepanipyrim, pyrimethanil and 3-(5-fluoro-3,3,4,4-tetramethyl-3,4-dihydroisoquinolin-1-yl)quinoline.

(8) Inhibitors of ATP production, for example fentin acetate, fentin chloride, fentin hydroxide and silthiofam.

(9) Cell wall synthesis inhibitors, for example benthiavalicarb, dimethomorph, flumorph, iprovalicarb, mandipropamid, polyoxins, polyoxorim, validamycin A and valifenalate.

(10) Lipid and membrane synthesis inhibitors, for example biphenyl, chloroneb, dicloran, edifenphos, etridiazole, iodocarb, iprobenfos, isoprothiolane, propamocarb, propamocarb hydrochloride, prothiocarb, pyrazophos, quintozene, tecnazene and tolclofos-methyl.

(11) Melanin biosynthesis inhibitors, for example carpropamid, diclocymet, fenoxanil, fthalide, pyroquilon, tricyclazole and 2,2,2-trifluoroethyl {3-methyl-1-[(4-methylbenzoyl)amino]butan-2-yl}carbamate.

(12) Nucleic acid synthesis inhibitors, for example benalaxyl, benalaxyl-M (kiralaxyl), bupirimate, clozylacon, dimethirimol, ethirimol, furalaxyl, hymexazol, metalaxyl, metalaxyl-M (mefenoxam), ofurace, oxadixyl and oxolinic acid.

(13) Signal transduction inhibitors, for example chlozolinate, fenpiclonil, fludioxonil, iprodione, procymidone, quinoxyfen and vinclozolin.

(14) Decouplers, for example binapacryl, dinocap, ferimzone, fluazinam and meptyldinocap.

(15) Further compounds, for example benthiazole, bethoxazin, capsimycin, carvone, chinomethionat, pyriofenone (chlazafenone), cufraneb, cyflufenamid, cymoxanil, cyprosulfamide, dazomet, debacarb, dichlorophen, diclomezine, difenzoquat, difenzoquat methylsulfate, diphenylamine, ecomat, fenpyrazamine, flumetover, fluoromide, flusulfamide, flutianil, fosetyl-aluminum, fosetyl-calcium, fosetyl-sodium, hexachlorobenzene, irumamycin, methasulfocarb, methyl isothiocyanate, metrafenon, mildiomycin, natamycin, nickel dimethyldithiocarbamate, nitrothal-isopropyl, octhilinone, oxamocarb, oxyfenthiin, pentachlorophenol and salts thereof, phenothrin, phosphoric acid and salts thereof, propamocarb-fosetylate, propanosine-sodium, proquinazid, pyrimorph, (2E)-3-(4-tert-butylphenyl)-3-(2-chloropyridin-4-yl)-1-(morpholin-4-yl)prop-2-en-1-one, (2Z)-3-(4-tert-butylphenyl)-3-(2-chloropyridin-4-yl)-1-(morpholin-4-yl)prop-2-en-1-one, pyrrolnitrin, tebufloquin, tecloftalam, tolnifanid, triazoxide, trichlamide, zarilamide, (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-methoxyphenoxy)-3,3-dimethylbutan-2-yl 1H-imidazole-1-carboxylate, 2,3,5,6-tetrachloro-4-(methylsulfonyl)pyridine, 2,3-dibutyl-6-chlorothieno[2,3-d]pyrimidin-4(3H)-one, 2,6-dimethyl-1H,5H-[1,4]dithiino[2,3-c:5,6-c′]dipyrrole-1,3,5,7(2H,6H)-tetrone, 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, 2-butoxy-6-iodo-3-propyl-4H-chromen-4-one, 2-chloro-5-[2-chloro-1-(2,6-difluoro-4-methoxyphenyl)-4-methyl-1H-imidazol-5-yl]pyridine, 2-phenylphenol and salts thereof, 3-(4,4,5-trifluoro-3,3-dimethyl-3,4-dihydroisoquinolin-1-yl)quinoline, 3,4,5-trichloropyridine-2,6-dicarbonitrile, 3-[5-(4-chlorophenyl)-2,3-dimethyl-1,2-oxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-amino-1,3,4-thiadiazole-2-thiol, 5-chloro-N′-phenyl-N′-(prop-2-yn-1-yl)thiophene-2-sulfonohydrazide, 5-fluoro-2-[(4-fluorobenzyl)oxy]pyrimidin-4-amine, 5-fluoro-2-[(4-methylbenzyl)oxy]pyrimidin-4-amine, 5-methyl-6-octyl[1,2,4]triazolo[1,5-a]pyrimidin-7-amine, ethyl (2Z)-3-amino-2-cyano-3-phenyl prop-2-enoate, N′-(4-{[3-(4-chlorobenzyl)-1,2,4-thiadiazol-5-yl]oxy}-2,5-dimethylphenyl)-N-ethyl-N-methylimidoformamide, N-(4-chlorobenzyl)-3-[3-methoxy-4-(prop-2-yn-1-yloxy)phenyl]propanamide, N-[(4-chlorophenyl)(cyano)methyl]-3-3-methoxy-4-(prop-2-yn-1-yloxy)phenyl]propanamide, N-[(5-bromo-3-chloropyridin-2-yl)methyl]-2,4-dichloropyridine-3-carboxamide, N-[(5-bromo-3-chloropyridin-2-yl)methyl]-2,4-dichloropyridine-3-carboxamide, N-[1-(5-bromo-3-chloropyridin-2-yl)ethyl]-2-fluoro-4-iodopyridine-3-carboxamide, N-{(E)-[(cyclopropylmethoxy)imino][6-(difluoromethoxy)-2,3-difluorophenyl]methyl}-2-phenylacetamide, N-{(Z)-[(cyclopropylmethoxy)imino][6-(difluoromethoxy)-2,3-difluorophenyl]methyl}-2-phenylacetamide, N′-{4-[3-tert-butyl-4-cyano-1,2-thiazol-5-yl)oxy]-2-chloro-5-methylphenyl}-N-ethyl-N-methylimidoformamide, N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, N-methyl-2-(1-{[5-methyl-3-(trifluoromethyl)-1H-pyrazol-1-yl]acetyl}piperidin-4-yl)-N-[(1 R)-1,2,3,4-tetrahydronaphthalen-1-yl]-1,3-thiazole-4-carboxamide, N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, pentyl {6-[({[(1-methyl-1H-tetrazol-5-yl)(phenyl)methylidene]amino}oxy)methyl]pyridin-2-yl}carbamate, phenazine-1-carboxylic acid, quinolin-8-ol, quinolin-8-ol sulfate (2:1) and tert-butyl {6-[({[(1-methyl-1H-tetrazol-5-yl)(phenyl)methylene]amino}oxy)methyl]pyridin-2-yl}carbamate.

(16) Further compounds, for example 1-methyl-3-(trifluoromethyl)-N-[2′-(trifluoromethyl)biphenyl-2-yl]-1H-pyrazole-4-carboxamide, N-(4′-chlorobiphenyl-2-yl)-3-(difluoromethyl)-1-methyl-1H-pyrazole-4-carboxamide, N-(2′,4′-dichlorobiphenyl-2-yl)-3-(difluoromethyl)-1-methyl-1H-pyrazole-4-carboxamide, 3-(difluoromethyl)-1-methyl-N-[4′-(trifluoromethyl)biphenyl-2-yl]-1H-pyrazole-4-carboxamide, N-(2′,5′-difluorobiphenyl-2-yl)-1-methyl-3-(trifluoromethyl)-1H-pyrazole-4-carboxamide, 3-(difluoromethyl)-1-methyl-N-[4′-(prop-1-yn-1-yl)biphenyl-2-yl]-1H-pyrazole-4-carboxamide, 5-fluoro-1,3-dimethyl-N-[4′-(prop-1-yn-1-yl)biphenyl-2-yl]-1H-pyrazole-4-carboxamide, 2-chloro-N-[4′-(prop-1-yn-1-yl)biphenyl-2-yl]pyridine-3-carboxamide, 3-(difluoromethyl)-N-[4′-(3,3-dimethylbut-1-yn-1-yl)biphenyl-2-yl]-1-methyl-1H-pyrazole-4-carboxamide, N-[4′-(3,3-dimethylbut-1-yn-1-yl)biphenyl-2-yl]-5-fluoro-1,3-dimethyl-1H-pyrazole-4-carboxamide, 3-(difluoromethyl)-N-(4′-ethynylbiphenyl-2-yl)-1-methyl-1H-pyrazole-4-carboxamide, N-(4′-ethynylbiphenyl-2-yl)-5-fluoro-1,3-dimethyl-1H-pyrazole-4-carboxamide, 2-chloro-N-(4′-ethynylbiphenyl-2-yl)pyridine-3-carboxamide, 2-chloro-N-[4′-(3,3-dimethylbut-1-yn-1-yl)biphenyl-2-yl]pyridine-3-carboxamide, 4-(difluoromethyl)-2-methyl-N-[4′-(trifluoromethyl)biphenyl-2-yl]-1,3-thiazole-5-carboxamide, 5-fluoro-N-[4′-(3-hydroxy-3-methylbut-1-yn-1-yl)biphenyl-2-yl]-1,3-dimethyl-1H-pyrazole-4-carboxamide, 2-chloro-N-[4′-(3-hydroxy-3-methylbut-1-yn-1-yl)biphenyl-2-yl]pyridine-3-carboxamide, 3-(difluoromethyl)-N-[4′-(3-methoxy-3-methylbut-1-yn-1-yl)biphenyl-2-yl]-1-methyl-1H-pyrazole-4-carboxamide, 5-fluoro-N-[4′-(3-methoxy-3-methylbut-1-yn-1-yl)biphenyl-2-yl]-1,3-dimethyl-1H-pyrazole-4-carboxamide, 2-chloro-N-[4′-(3-methoxy-3-methylbut-1-yn-1-yl)biphenyl-2-yl]pyridine-3-carboxamide, (5-bromo-2-methoxy-4-methylpyridin-3-yl)(2,3,4-trimethoxy-6-methylphenyl)methanone, N-[2-(4-{[3-(4-chlorophenyl)prop-2-yn-1-yl]oxy}-3-methoxyphenyl)ethyl]-N2-(methylsulfonyl)valinamide, 4-oxo-4-[(2-phenylethyl)amino]butanoic acid and but-3-yn-1-yl{6-[({[(Z)-(1-methyl-1H-tetrazol-5-yl)(phenyl)methylene]amino}oxy)methyl]pyridin-2-yl}carbamate.

Examples of bactericides include the following: bronopol, dichlorophen, nitrapyrin, nickel dimethyldithiocarbamate, kasugamycin, octhilinon, furancarboxylic acid, oxytetracycline, probenazole, streptomycin, tecloftalam, copper sulfate and other copper preparations.

Examples of insecticides, acaricides and nematicides include the following:

(1) Acetylcholinesterase (AChE) inhibitors, such as carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

(2) GABA-gated chloride channel antagonists, for example cyclodiene-organochlorines, e.g. chlordane and endosulfan; or phenylpyrazoles (fiproles), e.g. ethiprole and fipronil.

(3) Sodium channel modulators/voltage-gated sodium channel blockers, for example pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R) isomers], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans isomer], prallethrin, pyrethrins (pyrethrum), resmethrin, silafluofen, tefluthrin, tetramethrin, tetramethrin [(1R) isomers)], tralomethrin and transfluthrin; or DDT; or methoxychlor.

(4) Nicotinergic acetylcholine receptor (nAChR) agonists, for example neonicotinoids, e.g. acetamiprid, clothianidin, dinotefuran, imidacloprid, nitenpyram, thiacloprid and thiamethoxam; or nicotine.

(5) Allosteric activators of the nicotinergic acetylcholine receptor (nAChR), for example spinosyns, e.g. spinetoram and spinosad.

(6) Chloride channel activators, for example avermectins/milbemycins, e.g. abamectin, emamectin benzoate, lepimectin and milbemectin.

(7) Juvenile hormone imitators, for example juvenile hormone analogs e.g. hydroprene, kinoprene and methoprene; or fenoxycarb; or pyriproxyfen.

(8) Active ingredients with unknown or nonspecific mechanisms of action, for example alkyl halides, e.g. methyl bromide and other alkyl halides; or chloropicrin; or sulfuryl fluoride; or borax; or tartar emetic.

(9) Selective antifeedants, for example pymetrozine; or flonicamid.

(10) Mite growth inhibitors, for example clofentezine, hexythiazox and diflovidazin; or etoxazole.

(11) Microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1Fa, Cry2Ab, mCry3A, Cry3Ab, Cry3Bb, Cry34/35Ab1.

(12) Oxidative phosphorylation inhibitors, ATP disruptors, for example diafenthiuron; or organotin compounds, e.g. azocyclotin, cyhexatin and fenbutatin oxide; or propargite; or tetradifon.

(13) Oxidative phosphorylation decouplers that interrupt the H proton gradient, for example chlorfenapyr, DNOC and sulfluramid.

(14) Nicotinergic acetylcholine receptor antagonists, for example bensultap, cartap hydrochloride, thiocyclam, and thiosultap-sodium.

(15) Chitin biosynthesis inhibitors, type 0, for example bistrifluron, chlorfluazuron, diflubenzuron, flucycloxuron, flufenoxuron, hexaflumuron, lufenuron, novaluron, noviflumuron, teflubenzuron and triflumuron.

(16) Chitin biosynthesis inhibitors, type 1, for example buprofezin.

(17) Molting disruptors, dipteran, for example cyromazine.

(18) Ecdysone receptor agonists, for example chromafenozide, halofenozide, methoxyfenozide and tebufenozide.

(19) Octopaminergic agonists, for example amitraz.

(20) Complex-Ill electron transport inhibitors, for example hydramethylnon; or acequinocyl; or fluacrypyrim.

(21) Complex-I electron transport inhibitors, for example METI acaricides, e.g. fenazaquin, fenpyroximate, pyrimidifen, pyridaben, tebufenpyrad and tolfenpyrad; or rotenone (Derris).

(22) Voltage-dependent sodium channel blockers, for example indoxacarb; or metaflumizone.

(23) Inhibitors of acetyl-CoA carboxylase, for example tetronic and tetramic acid derivatives, e.g. spirodiclofen, spiromesifen and spirotetramat.

(24) Complex-IV electron transport inhibitors, for example phosphines, e.g. aluminum phosphide, calcium phosphide, phosphine and zinc phosphide; or cyanide.

(25) Complex-11 electron transport inhibitors, for example cyenopyrafen.

(26) Ryanodine receptor effectors, for example diamides, e.g. chlorantraniliprole and flubendiamide.

Further active ingredients with an unknown mechanism of action, for example amidoflumet, azadirachtin, benclothiaz, benzoximate, bifenazate, bromopropylate, chinomethionat, cryolite, cyantraniliprole (Cyazypyr), cyflumetofen, dicofol, diflovidazin, fluensulfone, flufenerim, flufiprole, fluopyram, fufenozide, imidaclothiz, iprodione, pyridalyl, pyrifluquinazon and iodomethane; and additionally preparations based on Bacillus firmus (I-1582, BioNeem, Votivo).

Examples of herbicides include:

acetochlor, acibenzolar, acibenzolar-S-methyl, acifluorfen, acifluorfen-sodium, aclonifen, alachlor, allidochlor, alloxydim, alloxydim-sodium, ametryne, amicarbazone, amidochlor, amidosulfuron, aminocyclopyrachlor, aminocyclopyrachlor-potassium, aminocyclopyrachlor-methyl, aminopyralid, amitrole, ammonium sulfamate, ancymidol, anilofos, asulam, atrazine, aviglycine, azafenidin, azimsulfuron, aziprotryne, beflubutamid, benazolin, benazolin-ethyl, bencarbazone, benfluralin, benfuresate, bensulide, bensulfuron, bensulfuron-methyl, bentazone, benzfendizone, benzobicyclon, benzofenap, benzofluor, benzoylprop, benzyladenine, bicyclopyrone, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbaryl, carbetamide, carfentrazone, carfentrazone-ethyl, carvone, chlorocholine chloride, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat-chloride, chlornitrofen, 4-chlorophenoxyacetic acid, chlorophthalim, chlorpropham, chlorthal-dimethyl, chlortoluron, chlorsulfuron, cinidon, cinidon-ethyl, cinmethylin, cinosulfuron, clethodim, clodinafop, clodinafop-propargyl, clofencet, clomazone, clomeprop, cloprop, clopyralid, cloransulam, cloransulam-methyl, cloxyfonac, cumyluron, cyanamide, cyanazine, cyclanilide, cycloate, cyclosulfamuron, cycloxydim, cycluron, cyhalofop, cyhalofop-butyl, cyperquat, cyprazine, cyprazole, cytokinine, 2,4-D, 2,4-DB, daimuron/dymron, dalapon, daminozide, dazomet, n-decanol, desmedipham, desmetryn, detosyl-pyrazolate (DTP), diallate, diaminozide, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, diisopropylnaphthalene, dipropetryn, diquat, diquat-dibromide, dithiopyr, diuron, DNOC, eglinazine-ethyl, endothal, EPTC, esprocarb, ethalfluralin, ethametsulfuron, ethametsulfuron-methyl, ethyl naphthylacetate, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoprop, fenoxaprop, fenoxaprop-P, fenoxaprop-ethyl, fenoxaprop-P-ethyl, fenoxasulfone, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, flurochloridone, fluroxypyr, fluroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, glufosinate-ammonium, glufosinate-P, glufosinate-P-ammonium, glufosinate-P-sodium, glyphosate, glyphosate-isopropylammonium, H-9201, i.e. 0-(2,4-dimethyl-6-nitrophenyl) O-ethyl isopropylphosphoramidothioate, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapyr, imazapyr-isopropylammonium, imazaquin, imazaquin-ammonium, imazethapyr, imazethapyr-ammonium, imazosulfuron, inabenfide, indanofan, indaziflam, indoleacetic acid (IAA), 4-indol-3-ylbutyric acid (IBA), iodosulfuron, iodosulfuron-methyl-sodium, iofensulfuron, iofensulfuron-sodium, ioxynil, ipfencarbazone, isocarbamid, isopropalin, isoproturon, isouron, isoxaben, isoxachlortole, isoxaflutole, isoxapyrifop, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat-chloride, mesosulfuron, mesosulfuron-methyl, mesotrione, methabenzthiazuron, metam, metamifop, metamitron, metazachlor, metazasulfuron, methazole, methiopyrsulfuron, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monosulfuron ester, monuron, MT-128, i.e. 6-chloro-N-[(2E)-3-chloroprop-2-en-1-yl]-5-methyl-N-phenylpyridazin-3-amine, MT-5950, i.e. N-[3-chloro-4-(1-methylethyl)phenyl]-2-methylpentanamide, NGGC-011, 1-naphthylacetic acid (NAA), naphthylacetamide (NAAm), 2-naphthoxyacetic acid, naproanilide, napropamide, naptalam, NC-310, i.e. 4-(2,4-dichlorobenzoyl)-1-methyl-5-benzyloxypyrazole, neburon, nicosulfuron, nipyraclofen, nitralin, nitrofen, nitroguaiacolate, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazole, paraquat, paraquat dichloride, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazole, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, secbumeton, sethoxydim, siduron, simazine, simetryn, SN-106279, i.e. methyl (2R)-2-({7-[2-chloro-4-(trifluoromethyl)phenoxy]-2-naphthyl}oxy)propanoate, sulcotrione, sulfallate (CDEC), sulfentrazone, sulfometuron, sulfometuron-methyl, sulfosate (glyphosate-trimesium), sulfosulfuron, SW-065, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, tebutam, tebuthiuron, tecnazene, tefuryltrione, tembotrione, tepraloxydim, terbacil, terbucarb, terbuchlor, terbumeton, terbuthylazine, terbutryne, thenylchlor, thiafluamide, thiazafluron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, triafamone, triallate, triasulfuron, triaziflam, triazofenamide, tribenuron, tribenuron-methyl, tribufos, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0862, i.e. 3,4-dichloro-N-{2-[(4,6-dimethoxypyrimidin-2-yl)oxy]benzyl}aniline, and the following compounds:

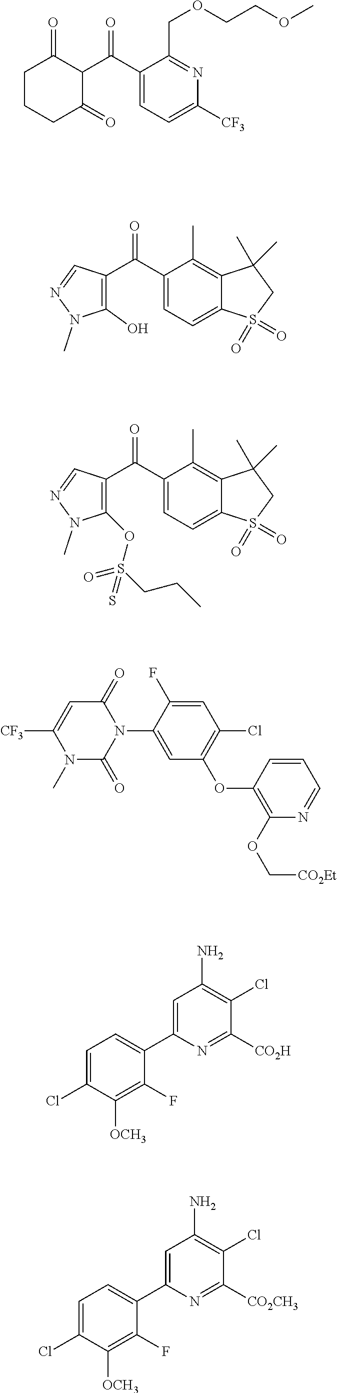 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=S)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl ; COc1c(Cl)ccc(-c2cc(N)c(Cl)c(C(=O)O)n2)c1F ; COC(=O)c1nc(-c2ccc(Cl)c(OC)c2F)cc(N)c1Cl]

Phytohormones control physiological reactions, such as growth, flowering rhythm, cell division and seed ripening. Examples of growth regulators include natural and synthetic plant hormones such as abscisic acid, benzyladenine, caprylic acid, decanol, indoleacetic acid, jasmonic acid and esters thereof, salicylic acid and esters thereof, gibberellic acid, kinetin and brassinosteroids.

Biological control agents are known to those skilled in the art and are described, for example, in “The Manual of Biocontrol Agents: A World Compendium, Copping, L. G., BCPC 2009”.

Examples of plant nutrients include customary inorganic or organic fertilizers for supplying plants with macro- and/or micronutrients.

Examples of repellents include diethyltolylamide, ethylhexanediol and butopyronoxyl.

The active substances are preferably selected from the group of the contact pesticides and/or the soil-active pesticides. Contact pesticides are understood to mean those pesticides that are not distributed within the target organism via the vascular system and therefore have a non-systemic mechanism of action. Soil-active pesticides are understood to mean those pesticides that, having been introduced onto or into the soil, display a long-term effect and are absorbed mainly via the root system of the plants treated.

Preferred contact pesticides are contact herbicides, contact insecticides or acaricides and/or protective fungicides.

Preferred soil-active pesticides are soil-active herbicides.

Examples of preferred contact herbicides are diphenyl ether herbicides, especially bifenox, or bipyridine herbicides, especially diquat or paraquat.

Examples of preferred soil-active herbicides are triazinone herbicides, especially metribuzin.

Examples of preferred contact insecticides or acaricides are pyrethroids, especially bifenthrin, cypermethrin, deltamethrin or esfenvalerad, or the carbamate insecticides, especially carbaryl, or the pyrazole insecticides, especially tebufenpyrad.

Examples of protective fungicides are phthalimide fungicides, especially captan or folpet, or the polychlorinated benzene compounds, especially chlorothalonil, or the dithiocarbamates, especially mancozeb.

The compounds of the formula I and compositions comprising one or more compounds of the formula I can be used in all customary formulation types, preferably in liquid compositions. In principle, however, the compounds may also be used in solid compositions.

Standard formulation forms for crop treatment compositions are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers-173, prepared by the FAO/WHO Joint Meeting on Pesticide Specifications, 2004, ISBN: 9251048576.

The active ingredient compositions may optionally contain action-improving adjuvants. An adjuvant in this context is a component which enhances the biological effect of the composition, without the component itself having any biological effect. Examples of adjuvants are penetrants, for example vegetable oils, for example rapeseed oil, sunflower oil, mineral oils, for example paraffin oils, alkyl esters of vegetable fatty acids, for example rapeseed oil methyl ester or soybean oil methyl ester, or alkanol alkoxylates and/or spreaders, for example alkylsiloxanes and/or salts, for example organic or inorganic ammonium or phosphonium salts, for example ammonium sulfate or diammonium hydrogenphosphate and/or retention promoters, for example dioctyl sulfosuccinate or hydroxypropylguar polymers and/or humectants, for example glycerol and/or fertilizers, for example ammonium-, potassium- or phosphorus-containing fertilizers and/or agents which promote sticking to the leaf surface.

Optionally, the active ingredient compositions may contain auxiliaries, preferably in combination with the abovementioned adjuvants. The auxiliaries may, for example, be extenders, solvents, spontaneity promoters, carriers, emulsifiers, dispersants, antifreezes, biocides and/or thickeners.

The active ingredient compositions are produced in a known manner, for example by mixing the active ingredients with auxiliaries, for example extenders, solvents and/or solid carriers and/or further auxiliaries, for example surfactants, preservatives, defoamers, functional polymers or additional adjuvants. The active ingredient compositions are produced either in suitable facilities or else before or during application.

Auxiliaries used may be those substances which are suitable for imparting particular properties, such as particular physical, technical and/or biological properties, to the formulation of the active ingredient or to the use forms prepared from these compositions (for example ready-to-use crop protection compositions such as spray liquids or seed dressing products).

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which may optionally also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

In principle, it is possible to use any suitable carriers. Useful carriers especially include: for example ammonium salts and natural rock flours such as kaolins, aluminas, talc, chalk, quartz, attapulgite, montmorillonite or diatomaceous earth, and synthetic rock flour such as finely divided silica, aluminum oxide and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, corn cobs and tobacco stalks.

It is also possible to use liquefied gaseous extenders or solvents. Especially suitable are those extenders or carriers which are gaseous at standard temperature and under standard pressure, for example aerosol propellants such as halohydrocarbons, or else butane, propane, nitrogen and carbon dioxide.

Examples of emulsifiers and/or foam formers, dispersants or wetting agents with ionic or nonionic properties, or mixtures of these surfactants, are salts of polyacrylic acid, salts of lignosulfonic acid, salts of phenolsulfonic acid or naphthalenesulfonic acid, polycondensates of ethylene oxide with fatty alcohols or with fatty acids or with fatty amines, with substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkyl sulfonates, alkyl sulfates, arylsulfonates, protein hydrolyzates, lignosulfite waste liquids and methyl cellulose. The presence of a surfactant is advantageous when one of the active ingredients and/or one of the inert carriers is insoluble in water and when application is effected in water.

Further auxiliaries which may be present in the compositions and the use forms derived therefrom are dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability. Foam generators or defoamers may additionally be present.

Preservatives used may be organic acids and esters thereof, for example ascorbic acid, ascorbyl palmitate, sorbate, benzoic acid, methyl and propyl 4-hydroxybenzoate, propionates, phenol, for example 2-phenylphenate, 1,2-benzisothiazolin-3-one, formaldehyde, sulfurous acid and salts thereof.

Suitable defoamers are fatty acid alkyl ester alkoxylates, organopolysiloxanes such as polydimethylsiloxanes and mixtures thereof with microfine, optionally silanized silica; perfluoroalkylphosphonates and -phosphinates, paraffins, waxes and microcrystalline waxes, and mixtures thereof with silanized silica. Also advantageous are mixtures of various foam inhibitors, for example those of silicone oil, paraffin oil and/or waxes.

The functional polymers which may be present in the active ingredient compositions of the invention are high molecular weight compounds of synthetic or natural origin having a molar mass of greater than 10,000. The functional polymers may act, for example, as an additional anti-drift agent or increase rain resistance.

In a further preferred embodiment of the invention, the active ingredient compositions of the invention comprise one or more further adjuvants as are usable in a known manner in aqueous agrochemical active ingredient compositions. These are preferably fatty amine ethoxylates, etheramine ethoxylates, alkyl betaines or amidoalkyl betaines, amine oxides or amidoalkylamine oxides, alkyl polyglycosides or copolymers of glycerol, coconut fatty acid and phthalic acid.

These adjuvants are known from the literature as adjuvants in aqueous pesticide compositions and are described, for example, in WO2009/029561.

In addition, the compositions and the use forms derived therefrom may also comprise, as additional auxiliaries, stickers such as carboxymethyl cellulose and natural and synthetic polymers in the form of powders, granules or latices, such as gum arabic, polyvinyl alcohol and polyvinyl acetate, or else natural phospholipids such as cephalins and lecithins and synthetic phospholipids. Further possible auxiliaries are mineral and vegetable oils.

It is possible if appropriate for still further auxiliaries to be present in the compositions and the use forms derived therefrom. Examples of such additives are fragrances, protective colloids, binders, adhesives, thickeners, thixotropic agents, penetrants, retention promoters, stabilizers, sequestrants, complexing agents, humectants, spreaders.

In general, the agrochemical substances can be combined with any solid or liquid additive which is commonly used for formulation purposes.

Useful retention promoters include all those substances which reduce dynamic surface tension, for example dioctyl sulfosuccinate, or increase viscoelasticity, for example hydroxypropylguar polymers.

Useful penetrants in the present context are all those substances which are typically used to improve the penetration of agrochemical substances into plants.

Penetrants are defined in this context by their ability to penetrate from the (generally aqueous) application liquid and/or from the spray deposit into the cuticle of the plant and hence increase the mobility of active ingredients in the cuticle. The method described in the literature (Baur et al., 1997, Pesticide Science 51, 131-152) can be used to determine this property. Examples include alcohol alkoxylates, for example coconut fat ethoxylate (10) or isotridecyl ethoxylate (12), fatty acid esters, for example rapeseed oil methyl ester or soya oil methyl ester, fatty amine alkoxylates, for example tallowamine ethoxylate (15) or ammonium salts and/or phosphonium salts, for example ammonium sulfate or diammonium hydrogenphosphate.

The active ingredient compositions of the invention preferably contain between 0.00001% and 98% by weight of active substance(s), more preferably between 0.01% and 95% by weight of active substance(s), more preferably between 0.5% and 90% by weight of active substance(s), based on the weight of the active ingredient composition.

The content of active substance(s) in the use forms (e.g. crop protection compositions) prepared from the active ingredient compositions can vary within wide limits. The concentration of the active substance(s) in the use forms, especially in the spray liquids, may typically be between 0.0000001% and 95% by weight of active substance(s), preferably between 0.00001% and 5% by weight of active substance(s), more preferably between 0.0001% and 1% by weight of active substance(s) and especially preferably between 0.001% and 1% by weight of active substance(s), based on the weight of the use form, especially of the spray liquid. Application is accomplished in a customary manner appropriate to the use forms.

The compositions are produced, for example, by mixing the components with one another in the particular ratios desired. If the active substance is a solid substance, it is generally used either in finely ground form or in the form of a solution or suspension in an organic solvent or water. If the active substance is liquid, there is frequently no need to use an organic solvent. It is also possible to use a solid active substance in the form of a melt. The temperatures can be varied within a particular range in the course of performance of the process. In general, working temperatures are between 0° C. and 80° C., preferably between 10° C. and 60° C.

According to the formulation type, the production of the active ingredient compositions of the invention is possible in various ways which are sufficiently well known to those skilled in the art. The procedure in the production may, for example, be to mix the N-alkenoyl-N-alkylglucamides of the formula I with one or more active substance(s) and optionally with auxiliaries. The sequence in which the components are mixed with one another is arbitrary. Useful equipment in the production is customary equipment which is used for production of agrochemical compositions.

The active ingredient compositions of the invention are preferably deployed to fields in the form of spray liquids. The spray liquids are produced by diluting concentrate formulations with a defined amount of water.

In a further preferred embodiment, the active ingredient compositions of the invention are in the form of spray liquids and contain 0.0000001% to 95% by weight, preferably 0.00001% to 5% by weight and more preferably 0.0001% to 1% by weight of active substance(s), and 0.001% to 3% by weight, preferably 0.005% to 1% by weight and especially preferably 0.05% to 0.2% by weight of the one or more N-alkenoyl-N-alkylglucamides of the formula I. The figures given are based on the overall spray liquid and, in the case of active substances which are used in the form of their water-soluble salts, on the amount of free acid, called the acid equivalent (a.e.).

The invention further relates to the use of the active ingredient compositions of the invention, in which the one or more active substances are one or more pesticides, and wherein preferably at least one of the one or more pesticides is a contact pesticide and/or a soil-active pesticide, for control and/or for combating of weeds, fungal diseases or insect infestation.

The N-alkenoyl-N-alkylglucamides of the formula I used in the active ingredient compositions of the invention are self-emulsifying and surprisingly are suitable as drift-reducing adjuvants for active ingredient compositions and bring about an effective increase in droplet size through a reduction in the fine droplet content in the spray. Furthermore, the N-alkenoyl-N-alkylglucamides of the formula I used in the active ingredient compositions of the invention surprisingly bring about a significant reduction in dynamic surface tension and are therefore suitable as adjuvants for improving wettability and adhesion when appyling the active ingredient compositions.

The combination of the above-described properties is surprising since it is known from the literature that surface-active substances, for example tallowamine ethoxylates, that lower dynamic surface tension normally exhibit an adverse effect on spray drift and lead to sprays with an elevated proportion of fine droplets (Hilz et al., Spray drift review: The extent to which a formulation can contribute to spray drift reduction, Crop Protection 44 (2013) 75-83).

“Self-emulsifying” N-alkenoyl-N-alkylglucamides in the context of the invention are understood to mean N-alkenoyl-N-alkylglucamides of the formula I which, because of their hydrophobic radical, have low water solubility and form stable homogeneous emulsions in the aqueous spray liquid. By contrast with conventional emulsions, where one or more water-insoluble hydrophobic substances (e.g. oils) are generally mixed with one or more emulsifiers, the N-alkenoyl-N-alkylglucamides that are “self-emulsifying” in accordance with the invention do not require any such auxiliaries. The “self-emulsifying” N-alkenoyl-N-alkylglucamides form stable homogeneous emulsions without the addition of additional emulsifiers or oils, and for the purpose require only slight to moderate agitation on mixing with water.

“Drift” in the context of the invention is understood to mean the effect that the spraying of the active ingredient composition forms small droplets which can be carried beyond the area to be treated, and can thus make the spraying less effective or even harmful to adjacent areas and crops.

In addition to the drift, relatively small droplets have a tendency to increased evaporization, which can also lead to reduced availability of the active ingredient in the target area.

In the context of the present invention, “drift reduction” or “reducing drift” are preferably understood to mean the reduction of the proportion of fine droplets having a diameter of <105 μm in the spray compared to the application of a composition which does not contain the N-alkenoyl-N-alkylglucamides of the formula I, preferably by at least 10% and more preferably by at least 25%.

It is known that the presence of particular substances in aqueous spray liquids increases the fine droplet content in the spray compared to aqueous spray liquids which do not contain these substances. If N-alkenoyl-N-alkylglucamides of the formula I are added to these spray liquids having an elevated fine droplet content, the relative reduction in drift may be even much higher than that specified above.

“Application” of an active ingredient composition in the form of a spray liquid containing one or more agrochemical substances in the context of the invention is understood to mean the application of an aqueous spray liquid containing one or more agrochemical substances to the species to be treated, for example the plants to be treated, and/or the locus thereof.

It is known from the literature that, given a timespan of relevance for the spray application of agrochemicals in aqueous dilution (called the surface age in the bubble pressure method) of 200 milliseconds, the value for dynamic surface tension in [mN/m] correlates with the wettability and adhesion on plants that are difficult to wet, such as barley (cereal). A value of 50 mN/m (at 20-21° C.) with respect to water (72.8 mN/m) results in an improvement in the adhesion from “zero adhesion” (0%) to about 50% (Baur P., Pontzen R.; 2007; Basic features of plant surface wettability and deposit formation and the impact of adjuvant; in R. E. Gaskin ed. Proceedings of the 8th International Symposium on Adjuvants for Agrochemicals; Publisher: International Society for Agrochemical Adjuvants (ISAA), Columbus, Ohio, USA).

Moreover, lowering of the surface tension, via improved penetration into fine pores in the soil, also contributes to promoting the efficacy of soil-active pesticides on application to dry arable land or to peaty soils.

In the context of the present invention, improved wetting characteristics are therefore defined via a reduction in the surface tension (measured with the KrUss BP2100 tensiometer, for determination of the dynamic surface tension by means of the bubble pressure method) of a 0.1% by weight aqueous solution at 200 ms, which is preferably lowered to less than 60 mN/m, and more preferably to less than 45 mN/m.

It has been found that the N-alkenoyl-N-alkylglucamides of the formula I in a 0.1% by weight aqueous solution lower the dynamic surface tension at 200 ms to less than 60 mN/m, preferably to less than 45 mN/m.

The invention therefore also relates to the use of one or more N-alkenoyl-N-alkylglucamides of the formula I for reducing drift and/or for improving wettability when appyling active ingredient compositions.

A preferred embodiment of the invention is the use of one or more N-alkenoyl-N-alkylglucamides of the formula I for reducing drift and/or for improving wettability when appyling active ingredient compositions as a tankmix additive. In this embodiment, the N-alkenoyl-N-alkylglucamides of the formula I are added to a spray liquid produced from a concentrated composition of the active ingredient formulation only directly prior to deployment.

The invention further provides a method of reducing drift and simultaneously improving wettability when appyling active ingredient compositions, wherein an aqueous spray liquid is sprayed onto the species to be treated, for example plants, and/or the locus thereof, wherein the spray liquid comprising active substance(s) contains one or more N-alkenoyl-N-alkylglucamides of the formula I in amounts of 0.001% to 5% by weight, more preferably of 0.005% to 3% by weight, especially preferably of 0.01% to 1% by weight and exceptionally preferably of 0.05% to 0.2% by weight, based in each case on the total weight of the spray liquid.

In many cases, a reduction in drift with simultaneous improvement in wettability occurs when the N-alkenoyl-N-alkylglucamides of the formula I are used.

The inventive use of the one or more N-alkenoyl-N-alkylglucamides of the formula I is preferably effected in ready-to-use active ingredient compositions in the form of spray liquids, in which case the amount of the one or more N-alkenoyl-N-alkylglucamides of the formula I in the spray liquid is preferably from 0.001% to 5% by weight, more preferably from 0.005% to 3% by weight, especially preferably from 0.01% to 1% by weight and exceptionally preferably from 0.05% to 0.2% by weight, based in each case on the total weight of the spray liquid. These spray liquids used in the inventive use preferably contain one or more agrochemical substances.

If an active ingredient composition comprises two or more N-alkenoyl-N-alkylglucamides of the formula I, the stated amount is understood to mean the total content of all N-alkenoyl-N-alkylglucamides of the formula I.

The radical definitions, value ranges and elucidations given above, in general terms or in ranges of preference, can be combined with one another as desired, i.e. including combinations between the particular ranges and ranges of preference.

The mechanism of action of the N-alkenoyl-N-alkylglucamides of the formula I used in accordance with the invention for reducing drift and/or for improving wettability when appyling active ingredient compositions is fundamentally independent of the nature of the active substance used. However, the selection of the optimal unsaturated alkylene radical R1 in the one or more N-alkenoyl-N-alkylglucamides of the formula I for maximum reduction of drift and simultaneous improvement in wettability on application can be affected by any substances present in the active ingredient compositions such as active ingredient salts or other salts, for example.

In the case of soil-active pesticides, of contact pesticides and of pesticides that exhibit strong phytotoxic action, significantly increased absorption of the active ingredient is frequently harmful to the efficacy thereof. Efforts are therefore being made to find additives that promote wetting and adhesion but only slightly increase the uptake of the active ingredient, if at all, such that only a small portion of the active ingredient is taken up into the leaf. It is known that short-chain alkylglucamides, for example Synergen® GA (N-methyl-N-octanoyl/decanoylglucamide), lead only to a slight increase in penetration, if any, via elevated diffusion through the cuticle of green plant parts or generally at the the plant level. Especially in the case of active ingredients having a molecular weight greater than 300 g/mol and a melting point of greater than 100° C., additives that greatly enhance uptake into the target organism are necessary for the biological efficacy thereof. But even in the case of active ingredients having smaller molecular weight, for example metribuzin (see table 6), the uptake of soil herbicides is increased by such additives (see table 6).

This is usually at the cost of prolonged action owing to a shift in the active ingredient dose from the soil to the plant parts above the ground, and hence poorer soil action. Examples of such penetration enhancers are additives comprising methylated vegetable oil (e.g. rapeseed oil methyl ester or soybean oil methyl ester) or particular alkoxylated alcohols.

It has been found that the N-alkenoyl-N-alkylglucamides of the formula I promote wetting and adhesion, but only very slightly increase the uptake of the active ingredient into the leaf, if at all.

In the context of the present invention, therefore, “additives that do not lead to enhanced uptake of an active ingredient” or “non-penetration-promoting additives” are understood to mean substances which, given comparable use concentrations and given comparable observation times, bring about uptake of active ingredients into the target organism at twice as slow or twice as low rate, preferably at a rate four times slower or lower and more preferably at a rate five times slower or lower than penetration-promoting additives comprising methylated vegetable oil.

A further preferred deployment of the invention is therefore the use of one or more self-emulsifying N-alkenoyl-N-alkylglucamides of the formula I for reducing drift and simultaneously improving wettability through reduction in the dynamic surface tension, which do not lead to enhanced uptake of an active agrochemical ingredient into the target organism when appyling active ingredient compositions.

The invention further provides a method of reducing drift and/or improving wettability when appyling active ingredient compositions, wherein an aqueous spray liquid is sprayed onto the species to be treated, for example plants, and/or the locus thereof, wherein the spray liquid comprising active substance(s) contains one or more N-alkenoyl-N-alkylglucamides of the formula I in amounts of 0.001% to 5% by weight, more preferably of 0.005% to 3% by weight, especially preferably of 0.01% to 1% by weight and exceptionally preferably of 0.05% to 0.2% by weight, based in each case on the total weight of the spray liquid.

The invention further provides a method of preventing enhanced uptake of an active agrochemical ingredient into the target organism when appyling active ingredient compositions, wherein an aqueous spray liquid is sprayed onto the species to be treated, for example plants, and/or the locus thereof, wherein the spray liquid comprising active substance(s) contains one or more N-alkenoyl-N-alkylglucamides of the formula I in amounts of 0.001% to 5% by weight, more preferably of 0.005% to 3% by weight, especially preferably of 0.01% to 1% by weight and exceptionally preferably of 0.05% to 0.2% by weight, based in each case on the total weight of the spray liquid.

The invention is illustrated hereinafter by examples, but these should in no way be regarded as a restriction.

The percentages stated hereinafter are percent by weight (% by weight), unless explicitly stated otherwise.

The raw materials used are:

| | |
|---|---|
| Synergen ® OS | anti-drift adjuvant from Clariant, |
| | based on a mixture of polyglycerol ester |
| | and rapeseed oil methyl ester |
| Hasten ® | modified vegetable oil adjuvant, from Willbur Ellis |
| Strikelock | methylated seed oil based adjuvant, from Winfield |
| Synergen ® GA | C8-10-N-glucamide (50% by weight |
| | active), from Clariant |
| AMS | ammonium sulfate, from Redox |
| Liberator ® | SC herbicide formulation with 400 g/L flufenacet |
| | and 100 g/L diflufenican, from Bayer |
| Flint ™ 50WG | WG fungicide formulation with 50% |
| fungicide | trifloxystrobin, from Bayer |
| Ascra Xpro | EC fungicide formulation with 75 g/L |
| | fluoxastrobin, 150 g/L prothioconazole, |
| | 75 g/L trifloxystrobin, from Bayer |
| Affirm ® 095 SG | SG insecticide formulation with 95 g/kg |
| | emamectin benzoate, from Syngenta |
| Sencor ® SC | SC herbicide formulation with 600 g/L |
| | metribuzin, from Bayer |
| Genamin ® T 150 | tallowamine ethoxylate, from Clariant |
| Water | deionized water or tap water |

### EXAMPLE 1

### Preparation of the N-alkenoyl-N-alkylglucamides of the Invention

Dodec-9-enoyl-N-methylglucamide was prepared from a commercially available dodec-9-enoylcarboxylic acid methyl ester by reaction with N-methylglucamine in propylene glycol as described in WO 92/06073. This gave a mixture which, as well as 90% by weight of the dodec-9-enoyl-N-methylglucamide of the formula (I) with R1=undec-8-enoyl radical and R2=CH₃, also contained 10% by weight of propylene glycol from the reaction mixture.

The C13-15-alkenoyl-N-methylglucamide was prepared from a commercially available unsaturated C13-15-carboxylic acid methyl ester, mainly consisting of a complex mixture of mono- and polyunsaturated C13- and C15-alkenoyl methyl esters, by reaction with N-methylglucamine in propylene glycol as described in WO 92/06073. This gave a mixture which, as well as 90% by weight of the unsaturated C13-15-alkenoyl-N-methylglucamide of the formula (I) with R1=mono- and polyunsaturated C12-14-alkenyl radical and R2=CH₃, also contained 10% by weight of propylene glycol from the reaction mixture.

Analogously, for comparison, the following non-inventive N-alkanoyl-N-methylglucamides were prepared from the corresponding linear saturated alkylcarboxylic acid methyl esters: C₈-C₁₄-alkanoyl-N-methylglucamide, dodecanoyl-N-methylglucamide, C₁₂-C₁₄-alkanoyl-N-methylglucamide.

### EXAMPLE 2

### Production of Sprya Liquids with N-alkenoyl-N-alkylglucamides

The composition of spray liquids A1-A18 is specified hereinafter. These spray liquids are produced by mixing the various test substances in water, and the appearance and stability of the spray liquid were assessed after 24 h.

TABLE 1

| | | Amount | | |
|---|---|---|---|---|
| Spray liquid | Alkylglucamide | [% by wt.] | Appearance | Stability |
| A1 | Dodec-9-enoyl-N- | 0.05 | cloudy | stable |
| | methylglucamide | | homogeneous | |
| | | | emulsion | |
| A2 | Dodec-9-enoyl-N- | 0.1 | cloudy | stable |
| | methylglucamide | | homogeneous | |
| | | | emulsion | |
| A3 | Dodec-9-enoyl-N- | 0.2 | cloudy | stable |
| | methylglucamide | | homogeneous | |
| | | | emulsion | |
| A4 | C13-15-alkenoyl- | 0.05 | cloudy | stable |
| | N- | | homogeneous | |
| | methylglucamide | | emulsion | |
| A5 | C13-15-alkenoyl- | 0.1 | cloudy | stable |
| | N- | | homogeneous | |
| | methylglucamide | | emulsion | |
| A6 | C13-15-alkenoyl- | 0.2 | cloudy | stable |
| | N- | | homogeneous | |
| | methylglucamide | | emulsion | |
| A7 (non- | dodecanoyl-N- | 0.05 | flocculation | phase |
| inventive) | methylglucamide | | | separation |
| A8 (non- | dodecanoyl-N- | 0.1 | flocculation | phase |
| inventive) | methylglucamide | | | separation |
| A9 (non- | dodecanoyl-N- | 0.2 | flocculation | phase |
| inventive) | methylglucamide | | | separation |
| A10 (non- | C12/14-alkanoyl- | 0.05 | flocculation | phase |
| inventive) | N- | | | separation |
| | methylglucamide | | | |
| A11 (non- | C12/14-alkanoyl-N- | 0.1 | flocculation | phase |
| inventive) | methylglucamide | | | separation |
| A12 (non- | C8/14-alkanoyl-N- | 0.05 | flocculation | phase |
| inventive) | methylglucamide | | | separation |
| A13 (non- | C8/14-alkanoyl-N- | 0.1 | flocculation | phase |
| inventive) | methylglucamide | | | separation |
| A14 (non- | Genamin T 150 | 0.05 | clear solution | stable |
| inventive) | | | | |
| A15 (non- | Genamin T 150 | 0.1 | clear solution | stable |
| inventive) | | | | |
| A16 (non- | Genamin T 150 | 0.2 | clear solution | stable |
| inventive) | | | | |
| A17 (non- | Synergen GA | 0.1 | clear solution | stable |
| inventive) | | | | |
| A18 (non- | 9-decenoyl-N- | 0.1 | clear solution | stable |
| inventive) | glucamide | | | |

The inventive alkenoylglucamides (A1 to A6) are self-emulsifying in the spray liquid and form cloudy homogeneous emulsions that are stable over 24 h. By contrast, spray liquids comprising non-inventive saturated alkylglucamides (A7-A13) of similar chain length are not phase-stable and flocculate. Shorter-chain alkylglucamides, for example Synergen GA (C8/10 alkylglucamide) or 9-decenoyl-N-glucamide, are completely water-soluble and form a clear transparent spray liquid.

### EXAMPLE 3

### Dynamic Surface Tension

Dynamic surface tension was determined via the bubble pressure method (BP2100 tensiometer, Krüss). Given a timespan of relevance for the spray application of agrochemicals in aqueous dilution (called the surface age in the bubble pressure method) of 200 milliseconds (ms), the value for dynamic surface tension in [mN/m] correlates with the adhesion on plants that are difficult to wet, such as barley (cereal). A value of 50 mN/m (at 20-21° C.) with respect to water (72.8 mN/m) results in an improvement in the adhesion from “zero adhesion” (0%) to about 50% (Baur P., Pontzen R.; 2007; Basic features of plant surface wettability and deposit formation and the impact of adjuvant; in R. E. Gaskin ed. Proceedings of the 8th International Symposium on Adjuvants for Agrochemicals; Publisher: International Society for Agrochemical Adjuvants (ISAA), Columbus, Ohio, USA).

It is additionally known from the literature that surface-active substances, for example tallowamine ethoxylates, that lower dynamic surface tension normally exhibit an adverse effect on spray drift and lead to sprays with an elevated proportion of fine droplets (Hilz et al., Spray drift review: The extent to which a formulation can contribute to spray drift reduction, Crop Protection 44 (2013) 75-83).

Surprisingly, the N-alkenoyl-N-alkylglucamides of the invention, in spite of low dynamic surface tension, show excellent drift-reducing properties (see tables 4 and 5).

TABLE 2

| | Dynamic surface tension [mN/m] | | | | |
|---|---|---|---|---|---|
| | Amount | | | | |
| | % by | 20 | 50 | 100 | 200 |
| Test substance | wt. | ms | ms | ms | ms |
| Dodec-9-enoyl-N-methylglucamide | 0.03 | 70.0 | 66.1 | 61.9 | 58.6 |
| Dodec-9-enoyl-N-methylglucamide | 0.1 | 56.1 | 47.7 | 44.0 | 40.1 |
| Dodec-9-enoyl-N-methylglucamide | 0.3 | 44.5 | 38.5 | 36.0 | 33.4 |
| C13-15-alkenoyl-N-methyl- | 0.03 | 72.9 | 71.8 | 70.8 | 69.6 |
| glucamide | | | | | |
| C13-15-alkenoyl-N-methyl- | 0.1 | 70.6 | 67.4 | 62.3 | 58.0 |
| glucamide | | | | | |
| C13-15-alkenoyl-N-methyl- | 0.3 | 60.4 | 54.2 | 48.2 | 45.5 |
| glucamide | | | | | |
| dodecanoyl-N-methylglucamide | 0.03 | 70.6 | 67.3 | 63.0 | 57.9 |
| dodecanoyl-N-methylglucamide | 0.1 | 70.0 | 66.8 | 60.1 | 51.4 |
| dodecanoyl-N-methylglucamide | 0.3 | 67.0 | 61.9 | 58.8 | 48.7 |
| Genamin T 150 | 0.03 | 68.2 | 65.5 | 62.2 | 59.0 |
| Genamin T 150 | 0.1 | 64.3 | 60.3 | 57.5 | 54.5 |
| Genamin T 150 | 0.3 | 60.0 | 56.6 | 53.4 | 50.7 |
| Synergen OS | 0.3 | 71.5 | 69.0 | 67.4 | 65.8 |
| Synergen GA | 0.03 | 71.4 | 70.8 | 70.4 | 70.7 |
| Synergen GA | 0.1 | 69.0 | 65.5 | 63.1 | 61.5 |
| Synergen GA | 0.3 | 55.1 | 50.3 | 47.1 | 45.5 |
| dec-9-enoyl-N-methylglucamide | 0.06 | 70.2 | 68.9 | 67.6 | 67.4 |
| dec-9-enoyl-N-methylglucamide | 0.125 | 66.4 | 63.7 | 61.8 | 61.0 |
| dec-9-enoyl-N-methylglucamide | 0.25 | 59.7 | 56.6 | 55.5 | 54.1 |

The results show that both of the tested N-alkenoyl-N-alkylglucamides of the formula I lowered the dynamic surface tension to a distinctly greater degree and are therefore distinctly better wetting agents compared to the saturated C12 variant of dodecanoyl-N-methylglucamide (C12, saturated) with similar chain length. Especially for Dodec-9-enoyl-N-methylglucamide (C12, unsaturated), the lowering of the dynamic surface tension is particularly large. Compared to commercially used wetting agents as well, such as Genamin T 150 (tallowamine ethoxylate), Dodec-9-enoyl-N-methylglucamide shows considerably better wetting properties. The anti-drift adjuvant Synergen OS does not show any significant lowering of the dynamic surface tension. Moreover, the lowering of the dynamic surface tension, given comparable concentrations for Dodec-9-enoyl-N-methylglucamide, is surprisingly much greater than in the case of other glucamides, for example Synergen GA or 9-decenoyl-N-methylglucamide.

### EXAMPLE 4

### Production of Spray Liquids with Tankmix Partner Formulation and N-alkenoyl-N-alkylglucamides

The composition of spray liquids B1-B14 is specified hereinafter. These spray liquids are produced by mixing a tankmix partner, for example ammonium sulfate (AMS) or pesticide formulations, the test substance and water. Homogeneous spray solutions are obtained.

TABLE 3

| | | Amount of | | Amount |
|---|---|---|---|---|
| | | tankmix | | of test |
| Spray | Tankmix | partner | | substance |
| liquid | partner | [% by wt.] | Test substance | [% by wt.] |
| B1 (non- | AMS | 0.5 | — | — |
| inventive) | | | | |
| B2 | AMS | 0.5 | Dodec-9-enoyl-N- | 0.1 |
| | | | methylglucamide | |
| B3 | AMS | 0.5 | C13-15-alkenoyl-N- | 0.1 |
| | | | methylglucamide | |
| B4 (non- | Flint WG 50 | 0.015 | — | — |
| inventive) | | | | |
| B5 | Flint WG 50 | 0.015 | Dodec-9-enoyl-N- | 0.1 |
| | | | methylglucamide | |
| B6 | Flint WG 50 | 0.015 | C13-15-alkenoyl-N- | 0.1 |
| | | | methylglucamide | |
| B7 (non- | Liberator | 0.30 | — | — |
| inventive) | | | | |
| B8 | Liberator | 0.30 | Dodec-9-enoyl-N- | 0.1 |
| | | | methylglucamide | |
| B9 | Liberator | 0.30 | C13-15-alkenoyl-N- | 0.1 |
| | | | methylglucamide | |
| B10 (non- | Ascra Xpro | 1.25 | — | — |
| inventive) | | | | |
| B11 | Ascra Xpro | 1.25 | Dodec-9-enoyl-N- | 0.1 |
| | | | methylglucamide | |
| B12 | Ascra Xpro | 1.25 | C13-15-alkenoyl-N- | 0.1 |
| | | | methylglucamide | |
| B13 (non- | Affirm ®095 | 0.055 | — | — |
| inventive) | SG | | | |
| B14 | Affirm ®095 | 0.055 | Dodec-9-enoyl-N- | 0.1 |
| | SG | | methylglucamide | |

### EXAMPLE 5

### Measurement of Droplet Size Distribution

A Malvern Spraytec “real-time spray sizing system” was used to determine the droplet size distribution. For this purpose, the system (STP5311, Malvern Instruments GmbH, Heidelberg, Germany) was installed in a specially constructed spray cabin, with the option of being able to model real spray applications as carried out in practice with freely adjustable pressure for various hydraulic nozzles and freely adjustable distances (nozzle-target surface). The spray cabin can be darkened and all disruptive parameters can be eliminated. For the measurements, the ID(3)12002 (Lechler) injector nozzles were used. The pressure set was varied, and mean pressure was kept constant at 3 bar for the measurements reported hereinafter. The temperature and relative air humidity varied between 21.5° C. and 29° C. and between 33% and 56% respectively. In each test series, tap water was measured as internal standard.

The Spraytec measurement was made at the setting of 1 kHz, since measurements at 2.5 kHz or higher, and also other influencing parameters such as additional suction, were found to be negligible. The measurement in the spray was kept constant at a position with distances of exactly 29.3 cm from the nozzle and 0.4 cm from the perpendicular below the nozzle. The measurements were made within 5 seconds, and the mean of 6 repetitions is reported as the proportion by volume of the droplets having diameters <90 μm (“Vol 90”), <105 μm (“Vol 105”) and <150 μm (“Vol 150”) (percentage standard error 0.5%-2.5%).

As a further measurement parameter, the proportion by volume of the droplets having diameter <210 μm (“Vol 210”) was determined and expressed in relation to the proportion by volume of droplets having diameter <105 μm (“Vol 210 / Vol 105”). In addition, the percentage reduction in the proportion by volume of droplets having diameter <105 μm in the case of use of spray liquids containing the test substances was calculated in comparison to the use of tap water as internal standard (“Red 105”).

TABLE 4
Droplet size distribution for ID(3)12002 injector nozzle (at 3
bar) using spray liquids A1-A17 (for composition see table 1).

| | | Vol 90 | Vol 105 | Vol 150 | Vol 210/ | Red 105 |
|---|---|---|---|---|---|---|
| Spray liquid | Test substance | [% by vol.] | [% by vol.] | [% by vol.] | Vol 105 | [%] |
| water | — | 1.88 | 3.00 | 6.79 | 4.02 | 0.00 |
| A1 | dodec-9-enoyl-N- | 0.93 | 1.41 | 2.95 | 4.27 | 52.88 |
| | methylglucamide | | | | | |
| A2 | dodec-9-enoyl-N- | 0.89 | 1.33 | 2.94 | 5.14 | 55.79 |
| | methylglucamide | | | | | |
| A3 | dodec-9-enoyl-N- | 1.06 | 1.63 | 3.80 | 5.16 | 45.64 |
| | methylglucamide | | | | | |
| A4 | C13-15-alkenoyl-N- | 0.88 | 1.34 | 2.56 | 3.36 | 55.30 |
| | methylglucamide | | | | | |
| A5 | C13-15-alkenoyl-N- | 0.93 | 1.41 | 2.70 | 3.41 | 52.88 |
| | methylglucamide | | | | | |
| A6 | C13-15-alkenoyl-N- | 0.95 | 1.47 | 3.06 | 4.07 | 50.89 |
| | methylglucamide | | | | | |
| A7 (non- | dodecanoyl-N- | 1.00 | 1.45 | 2.69 | 3.14 | 51.47 |
| inventive) | methylglucamide | | | | | |
| A8 (non- | dodecanoyl-N- | 0.87 | 1.33 | 2.44 | 2.97 | 55.64 |
| inventive) | methylglucamide | | | | | |
| A9 (non- | dodecanoyl-N- | 1.45 | 2.27 | 4.72 | 3.63 | 24.38 |
| inventive) | methylglucamide | | | | | |
| A14 (non- | Genamin ® T 150 | 3.18 | 4.69 | 9.88 | 3.79 | −46.53 |
| inventive) | | | | | | |
| A15 (non- | Genamin ® T 150 | 3.26 | 5.29 | 12.87 | 4.49 | −65.26 |
| inventive) | | | | | | |
| A16 (non- | Genamin ® T 150 | 3.85 | 6.49 | 16.53 | 4.68 | −102.64 |
| inventive) | | | | | | |
| A17 (non- | Synergen ® OS | 0.88 | 1.30 | 2.36 | 3.16 | 56.70 |
| inventive) | | | | | | |

Typically, wetting agents, for example Genamin® T 150 (tallowamine ethoxylate, V14-V16), which reduce dynamic surface tension lead to a drastic increase in fine droplet content. The results show that both the N-alkenoyl-N-alkylglucamides tested, in addition to their excellent wetting properties, surprisingly bring about a reduction in the fine droplet content, comparable to the commercially available anti-drift adjuvant Synergen® OS. Especially surprising is the effect for dodec-9-enoyl-N-methylglucamide (C12, unsaturated), which, in spite of the particularly significant lowering of the dynamic surface tension, brings about a distinct reduction in the fine droplet content.

TABLE 5
Droplet size distribution for ID(3)12002 injector nozzle (at 3
bar) using spray liquids B1-B14 (for composition see table 3).

| | Test substance/ | Vol 90 | Vol 105 | Vol 150 | Vol 210/ | Red 105 |
|---|---|---|---|---|---|---|
| Spray liquid | tankmix partner | [% by vol.] | [% by vol.] | [% by vol.] | Vol 105 | [%] |
| water | — | 1.96 | 3.13 | 7.05 | 3.97 | 0.00 |
| B1 (non- | —/AMS | 1.78 | 2.85 | 6.55 | 4.14 | 6.25 |
| inventive) | | | | | | |
| B2 | dodec-9-enoyl-N- | 0.99 | 1.54 | 3.49 | 4.89 | 49.51 |
| | methylglucamide/ | | | | | |
| | AMS | | | | | |
| B3 | C13-15-alkenoyl-N- | 0.91 | 1.39 | 2.76 | 3.66 | 54.43 |
| | methylglucamide/ | | | | | |
| | AMS | | | | | |
| B4 (non- | —/Flint WG 50 | 0.81 | 1.28 | 2.74 | 3.77 | 58.94 |
| inventive) | | | | | | |
| B5 | dodec-9-enoyl-N- | 0.79 | 1.17 | 2.46 | 4.66 | 62.53 |
| | methylglucamide/Flint | | | | | |
| | WG 50 | | | | | |
| B6 | C13-15-alkenoyl-N- | 0.81 | 1.22 | 2.34 | 3.59 | 60.91 |
| | methylglucamide/Flint | | | | | |
| | WG 50 | | | | | |
| B7 (non- | —/Liberator | 1.44 | 2.23 | 4.82 | 3.94 | 28.55 |
| inventive) | | | | | | |
| B8 | dodec-9-enoyl-N- | 0.73 | 1.11 | 2.27 | 4.20 | 64.35 |
| | methylglucamide/ | | | | | |
| | Liberator | | | | | |
| B9 | C13-15-alkenoyl-N- | 0.77 | 1.20 | 2.51 | 4.06 | 61.67 |
| | methylglucamide/ | | | | | |
| | Liberator | | | | | |
| B10 (non- | —/Ascra Xpro | 2.53 | 4.40 | 11.63 | 5.05 | −38.10 |
| inventive) | | | | | | |
| B11 | dodec-9-enoyl-N- | 1.07 | 1.61 | 3.58 | 4.77 | 49.44 |
| | methylglucamide/ | | | | | |
| | Ascra Xpro | | | | | |
| B12 | C13-15-alkenoyl-N- | 1.02 | 1.58 | 3.74 | 5.21 | 50.58 |
| | methylglucamide/ | | | | | |
| | Ascra Xpro | | | | | |
| B13 (non- | —/Affirm ®095 SG | 1.21 | 1.89 | 4.09 | 3.87 | 41.08 |
| inventive) | | | | | | |
| B14 | dodec-9-enoyl-N- | 1.18 | 1.85 | 4.40 | 5.12 | 42.27 |
| | methylglucamide/ | | | | | |
| | Affirm ®095 SG | | | | | |

The results show that both N-alkenoyl-N-alkylglucamides, even in the presence of typical tankmix partners such as AMS or commercially available pesticide formulations, bring about a reduction in the fine droplet content.

### EXAMPLE 6

### Penetration Characteristics of Soil-Active or Contact Active Ingredients using the Example of Metribuzin and Test System for Measurement of the Enhancement of Penetration of Active Ingredients

Surfactants can affect the absorption of (active) ingredients through membranes such as skin, films or the plant cuticle. As a “finite-dose” application, it is known for the single administration or application of a solution, cream, gel etc. to a membrane that the absorption of active ingredient can be influenced by some additives such as surfactants even after wetting. This effect is independent of the interfacial effect in water, is often highly concentration-dependent and takes place for the most part after evaporation of water and any solvents present as a result of the interaction, for example, with active ingredient, membrane and environmental factors. For various surfactants, it is observed after addition to active ingredient preparations that the penetration of a particular active ingredient is promoted to an enormous degree by some surfactants, whereas others are entirely ineffective (Cronfeld, P., Lader, K., Baur, P. (2001). Classification of Adjuvants and Adjuvant Blends by Effects on Cuticular Penetration, Pesticide Formulations and Application Systems: Twentieth Volume, ASTM STP 1400, A. K. Viets, R. S. Tann, J. C. Mueninghoff, eds., American Society for Testing and Materials, West Conshohocken, Pa. 2001).

The potential of the test substances, which is independent of the surfactant action, to promote foliar absorption of active agrochemical ingredients was determined in membrane penetration tests with apple leaf cuticles using the example of metribuzin. In the case of soil-active pesticides, for example metribuzin, and in the case of particular contact pesticides, greatly increased uptake of the active ingredient is harmful for efficacy. Efforts are therefore being made to find additives that promote wetting but only slightly increase the uptake of the active ingredient, such that only a minimum portion of the active ingredient is taken up into the leaf.

It is known that short-chain alkylglucamides, for example Synergen® GA (N-methyl-N-octanoyl/decanoylglucamine), lead only to a slight increase in penetration, if any, via elevated diffusion through the cuticle of green plant parts or generally at the the plant level. Especially in the case of active ingredients having a molecular weight greater than 300 g/mol and the melting point of greater than 100° C., additives that greatly enhance uptake into the target organism are necessary for the biological efficacy thereof. But even in the case of active ingredients having smaller molecular weight, for example metribuzin (see table 6), the uptake of soil herbicides is increased by such additives (see table 6). This is usually at the cost of prolonged action owing to a shift in the active ingredient dose from the soil to plant parts above the ground, and hence poorer soil action. Examples of such penetration enhancers are additives comprising methylated vegetable oil (e.g. rapeseed oil methyl ester or soybean oil methyl ester) or particular alkoxylated alcohols. It has been found that the inventive N-alkenoyl-N-alkylglucamides of the formula I promote wetting and adhesion, but only very slightly increase the uptake of the active ingredient into the leaf, if at all.

The plant cuticle is a lipophilic solubility membrane (lipid membrane) without pores or holes, and the results described are also expected for other nonporous lipophilic solubility membranes with these or other electrolyte active ingredients. The principle of the method has been published (e.g. WO-A-2005/194844; Baur, 1997; Baur, Grayson and Schönherr 1999; Baur, Bodelon and Lowe, 2012), and only the specifics and differences in the method are elucidated hereinafter. The leaf cuticles were enzymatically isolated in the manner described in the literature from apple leaves of orchard trees in a commercial stone fruit growing facility near Frankfurt am Main in 2016. The stomata-free cuticles were first dried under air and then installed into stainless steel diffusion cells. After application to the original upper side of the leaf and evaporation of the test liquid, i.e. of the aqueous preparations of the active ingredients without or with the glucamide-containing spray liquids or comparative compositions, the diffusion cells were transferred into thermostatted blocks and charged with aqueous liquid. The water used to make up the aqueous test liquids was local tap water (of known composition). At regular intervals, aliquot samples were taken and the proportion of active ingredient penetrated was determined by HPLC. During the experiment, the temperature in the system (block, diffusion cells, liquids, etc.) and the air humidity above the spray deposit on the cuticle were known exactly and were monitored. In the experiment, relative air humidity was constant throughout at 56% relative air humidity (air over supersaturated calcium nitrate) at a constant 10° C. The analytical determination by means of HPLC (1290 Infinity, Agilent) was effected thereafter with a Kinetex column 30×2, 1 mm, 2.6 μ C18 100 A (Phenomenex), taking a 20 μL aliquot as injection volume at the specified times. In each case, the geometric mean values of the penetration for intact membranes at the mean measurement times are given. According to the variant (active ingredient×test additive/formulation), 7-8 repetitions were set up. The coefficient of variation was below 35%, which is a typical biological variability for penetration for numerous plants (Baur, 1997).

TABLE 6
Penetration of metribuzin (formulation of Sencor 600
SC with active ingredient concentration 2.25 g/L in spray
liquid) in the presence of various test substances

| | Mean penetration for metribuzin | | |
|---|---|---|---|
| | (amount of active ingredient | | |
| | 2.25 g/L) after time (n = 4-8) in % | | |
| | Amount % by | | |
| Test substance | wt. | 1 day | 2 days |
| — | — | 1.4 | 3.9 |
| Synergen ® GA | 0.25 | 5.1 | 4.9 |
| Synergen ® OS | 0.14 | 10.1 | 16.4 |
| dodec-9-enoyl-N-methylglucamide | 0.05 | 2.9 | 4.6 |
| dodec-9-enoyl-N-methylglucamide | 0.1 | 3.9 | 6.5 |
| dodec-9-enoyl-N-methylglucamide | 0.14 | 5.7 | 8.6 |
| C13-15-alkenoyl-N-methylglucamide | 0.05 | 3.6 | 4.3 |
| C13-15-alkenoyl-N-methylglucamide | 0.1 | 6.9 | 9.8 |
| C13-15-alkenoyl-N-methylglucamide | 0.14 | 6.7 | 11.7 |
| Strikelock ® | 0.05 | 17.1 | 24.7 |
| Strikelock ® | 0.1 | 23.6 | 42.7 |
| Strikelock | 0.14 | 31.8 | 55.6 |
| Hasten ® | 0.05 | 11.4 | 25.6 |
| Hasten ® | 0.1 | 39.1 | 58.4 |
| Hasten ® | 0.14 | 45.0 | 72.3 |

*10° C./56% rel. air humidity

The table shows that both N-alkenoyl-N-alkylglucamides, similarly to the short-chain alkylglucamide Synergen® GA (N-methyl-N-octanoyl/decanoylglucamine), by comparison with methylated vegetable oil-based products (Synergen® OS, Strikelock® and Hasten®), lead only to minor promotion of penetration. Given comparable concentrations, the uptake of the N-alkenoyl-N-alkylglucamides is lower by more than two times compared to Synergen OS, more than five times compared to Strikelock, and more than seven times compared to Hasten.

### EXAMPLE 7

The plant compatibility of dodec-9-enoyl-N-methylglucamide and C13-15-alkenoyl-N-methylglucamide at the abovementioned concentrations of 0.1, 0.3, 0.8, 1.0, 1.5, 2 and 3 g/L is just as good as that of Synergen GA and N-methyl-N-nonanoylglucamine. After application to indicator plants (described, for example, in DE102014018274A1), there were no necroses or other symptoms such as leaf curl-up or deformation in any case, whereas an ethoxylated lauryl alcohol at 1 g/L that was also tested caused distinct necroses within one day.

1. An active ingredient composition comprising
a) one or more active substances selected from the group consisting of pesticides, and
b) one or more N-alkenoyl-N-alkylglucamides of the formula I 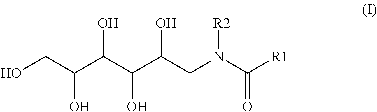
in which
R1 is a linear or branched alkenyl group which has 11 to 15 carbon atoms and has a non-terminal double bond or a plurality of conjugated or non-conjugated double bonds, and
R2 is an alkyl group having 1 to 4 carbon atoms.

2. (canceled)

3. The active ingredient composition as claimed in claim 1, wherein R1 has 12 to 14 carbon atoms.

4. The active ingredient composition as claimed in claim 3, wherein R1 is a dodecadienyl radical or a tetradecadienyl radical

5. The active ingredient composition as claimed in claim 1, wherein R1 is an undecenyl radical having a non-terminal double bond.

6. The active ingredient composition as claimed in claim 1, wherein R1 derives from a mixture of N-alkenyl acid methyl esters that comprises diunsaturated C₁₃-alkenyl acid methyl esters, monounsaturated C14-alkenyl acid methyl esters, monounsaturated C₁₅-alkenyl acid methyl esters and diunsaturated C₁₅-alkenyl acid methyl esters.

7. The active ingredient composition as claimed in claim 1, wherein R2 is methyl.

8. (canceled)

9. The active ingredient composition as claimed in claim 1, wherein at least one of the pesticides is a contact pesticide and/or a soil-active pesticide.

10. The active ingredient composition as claimed in claim 1, wherein it takes the form of a spray liquid and comprises 0.00001% to 5% by weight of active substance(s) and 0.001% to 3% by weight of the N-alkenoyl-N-alkylglucamide(s) of the formula I, where the stated amounts are based on the overall spray liquid.

11. An N-alkenoyl-N-alkylglucamide of the formula I 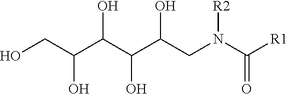
in which
R1 is an undec-8-enyl radical, a dodecadienyl radical, a tetradecadienyl radical, or wherein R1 derives from a mixture of N-alkenyl acid methyl esters that comprises diunsaturated C13-alkenyl acid methyl esters, monounsaturated C14-alkenyl acid methyl esters, monounsaturated C15-alkenyl acid methyl esters and diunsaturated C15-alkenyl acid methyl esters, and
R2 is an alkyl group having 1 to 4 carbon atoms.

12. The N-alkenoyl-N-alkylglucamide as claimed in claim 11, wherein R2 is methyl.

13. An adjuvant composition comprising
(A1) an N-alkenoyl-N-alkylglucamide of the formula I as claimed in claim 11, and
(A2) a cosolvent.

13-20. (canceled)

21. A method of reducing drift and of simultaneously improving wettability when appyling an active ingredient composition, comprising
a) one or more active substances selected from the group consisting of pesticides, and
b) one or more N-alkenoyl-N-alkylglucamides of the formula I 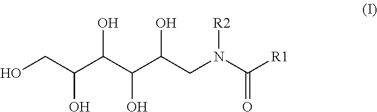
in which
R1 is a linear or branched alkenyl group which has 11 to 15 carbon atoms and has a non-terminal double bond or a plurality of conjugated or non-conjugated double bonds, and
R2 is an alkyl group having 1 to 4 carbon atoms,
wherein an aqueous spray liquid comprising the active ingredient composition is sprayed onto the species to be treated and/or the locus thereof, wherein the spray liquid comprises one or more N-alkenoyl-N-alkylglucamides of the formula I in amounts of 0.001% to 5% by weight, based on the total weight of the spray liquid.

22. The method as claimed in claim 21, in which at least one of the active substances is a pesticide, for control and/or for combating of weeds, fungal diseases or insect infestation.

23. The method as claimed in claim 21, wherein the active ingredient compositions comprise at least one contact pesticide and/or soil-active pesticide.

24. The method as claimed in claim 21, wherein the N-alkenoyl-N-alkylglucamide of the formula I is used for reducing drift and for improving wettability when appyling the active ingredient compositions, and does not lead to enhanced uptake of an active agrochemical ingredient into the target organism.
